(19) United States
(12) Patent Application Publication
(10) Pub. No.: US 2009/0012012 A1
(43) Pub. Date: Jan. 8, 2009

(54) Intratracheal administration of endothelin-suppressing agents for the treatment of respiratory disorders

(76) Inventors: Jerome Cantor, Brooklyn, NY (US); Tapan Bhavsar, Queens, NY (US); Sandra Reznik, Larchmont, NY (US)

Correspondence Address: Jerome Cantor, MD, 258 82nd St, Brooklyn, NY, 11209, US

(21) Appl. No.: 11/824,860

(22) Filed: Jul. 5, 2007

(51) Int. Cl. A61K 31/70; A61P 11/00

(52) U.S. Cl. 514/25

(57) ABSTRACT

The subject invention is directed to the treatment of respiratory disorders by intratracheal administration of an effective amount of an agent that suppresses the activity of endothelin. Such agents may take the form of: 1) an endothelin-converting enzyme (ECE) inhibitor such as phosphoramidon, or 2) an endothelin receptor antagonist such as bosentan, tezosentan, sitaxsentan, atrasentan, darusentan, clazosentan, or BQ-123. Respiratory disorders include emphysema, asthma, bronchitis, bronchiectasis, pneumonia, adult respiratory distress syndrome, neonatal respiratory distress syndrome, bronchopulmonary dysplasia, interstitial fibrosis, cystic fibrosis, persistent pulmonary hypertension of the newborn, and neoplasia. The treatment is intended for a variety of mammals, such as premature neonates to adult humans.

### RELATED APPLICATIONS

This application claims benefit of provisional applications Nos. 06/816,048 and 60/816,047, both filed on Jun. 26, 2006.

### SUMMARY OF THE INVENTION

The subject invention is directed to the treatment of respiratory disorders by intratracheal administration of an effective amount of an agent that suppresses the activity of endothelin. Such agents may take the form of: 1) an endothelin-converting enzyme (ECE) inhibitor such as phosphoramidon, or 2) an endothelin receptor antagonist such as bosentan, tezosentan, sitaxsentan, atrasentan, darusentan, clazosentan, or BQ-123. Respiratory disorders include emphysema, asthma, bronchitis, bronchiectasis, pneumonia, adult respiratory distress syndrome, neonatal respiratory distress syndrome, bronchopulmonary dysplasia, interstitial fibrosis, cystic fibrosis, persistent pulmonary hypertension of the newborn, and neoplasia. The treatment is intended for a variety of mammals, such as premature neonates to adult humans.

Administration of the ECE inhibitor or endothelin receptor antagonist may be performed by aerosol, which can be generated by a nebulizer, or by instillation. The ECE inhibitor or endothelin receptor antagonist may be administered alone, or with a carrier such as saline solution, DMSO, an alcohol, or water. The effective daily amount of the ECE inhibitor or endothelin receptor antagonist is from about 1 μg/kg to about 1 mg/kg of body weight.

### BRIEF DESCRIPTION OF FIGURES

FIG. 1: Animals receiving i.p. phosphoramidon (PHDN) and i.t. LPS had significantly less pulmonary inflammation than those given i.p. PBS and i.t. LPS.

FIG. 2: Animals receiving aerosolized phosphoramidon and i.t. LPS had significantly less pulmonary inflammation than those given aerosolized water and i.t. LPS.

FIG. 3: Animals receiving i.p. phosphoramidon and i.t. LPS had significantly fewer BALF neutrophils than those given i.p. PBS and i.t. LPS.

FIG. 4: Animals receiving aerosolized phosphoramidon and i.t. LPS had significantly less pulmonary inflammation than those given aerosolized water and i.t. LPS.

FIG. 5: Animals receiving i.p. phosphoramidon and i.t. LPS had a significantly lower percentage of TNFR1-positive BALF macrophages than those given i.p. PBS and i.t. LPS.

FIG. 6: Animals receiving aerosolized phosphoramidon and i.t. LPS had significantly less pulmonary inflammation than those aerosolized water and i.t. LPS.

FIG. 7: Animals receiving i.p. phosphoramidon and i.t. LPS had significantly fewer TUNEL-positive alveolar septal cells than those given i.p. PBS and i.t. LPS.

FIG. 8: Animals receiving aerosolized phosphoramidon and i.t. LPS had significantly fewer TUNEL-positive alveolar septal cells than those given aerosolized water and i.t. LPS alone.

### DETAILED DESCRIPTION OF THE INVENTION

The subject invention is directed to the treatment of respiratory disorders by intratracheal administration of an effective amount of an agent that suppresses the activity of endothelin. Such agents may take the form of: 1) an ECE inhibitor such as phosphoramidon, or 2) an endothelin receptor antagonist such as bosentan, tezosentan, sitaxsentan, atrasentan, darusentan, clazosentan, or BQ-123. Respiratory disorders include emphysema, asthma, bronchitis, bronchiectasis, pneumonia, adult respiratory distress syndrome, neonatal respiratory distress syndrome, bronchopulmonary dysplasia, interstitial fibrosis, cystic fibrosis, persistent pulmonary hypertension of the newborn, and neoplasia. The treatment is intended for a variety of mammals, such as premature neonates to adult humans. All agents may be derived from a natural source, produced by a bioprocess (such as fermentation), or chemically synthesized.

Administration of the ECE inhibitor or endothelin receptor antagonist, may be performed by aerosol, which can be generated by a nebulizer, or by instillation. The ECE inhibitor or endothelin receptor antagonist may be administered alone, or with a carrier such as saline solution, DMSO, an alcohol, or water. The effective daily amount of the ECE inhibitor or endothelin receptor antagonist is from about 1 μg/kg to about 1 mg/kg of body weight.

The amount of ECE inhibitor or endothelin receptor antagonist intratracheally administered daily to a human being may vary from about 1 μg/kg to about 1 mg/kg of body weight. Preferably, the daily amount is from about 10 μg/kg to about 100 μg/kg, for example about 50 μg/kg body weight of the human being treated (daily). The intratracheal ECE inhibitor or endothelin receptor antagonist may be administered in any of the methods well known to those skilled in the art. For example, it may be administered in the form of an aerosol or may be administered by instillation. If administered in the form of an aerosol, a nebulizer is used to produce the ECE inhibitor or endothelin receptor antagonist in aerosol form (See for example U.S. Pat. Nos. 4,649,911 and 4,119,096).

Typically, the ECE inhibitor or endothelin receptor antagonist is administered in a pharmaceutically acceptable carrier. Such examples include saline solution, DMSO, an alcohol, or water. Such carriers are well known in the art, and the specific carriers employed may be varied depending upon factors such as size of the subject being treated, treatment dose, and the like.

Further, the time over which the ECE inhibitor or endothelin receptor antagonist is administered may vary as is well known in the art to achieve the desired results. For example, the ECE inhibitor or endothelin receptor antagonist may be administered as an aerosol from about 10 minutes to about 1 hour per treatment regimen, 3 times daily, or until the desired daily dosage is fully administered.

### BACKGROUND OF THE INVENTION

### Introduction

Phosphoramidon, a zinc metalloproteinase inhibitor, blocks the activity of endothelin converting endopeptidase (ECE), thereby preventing formation of endothelin-1 (ET-1), a potent vasoconstrictor peptide (Hisaki et al., 1994; Forni et al., 2005; Donahue et al., 2006; Matsumaru et al., 1998). As a result, phosphoramidon has become a valuable tool in the study of vascular disease (Park et al., 2001; Aguilar et al., 2000) and may also be useful in limiting ischemic injury (Hassan et al., 1997; Keller et al., 1996).

Recently, phosphoramidon has been shown to prevent lipopolysaccharide (LPS)-induced preterm delivery in mice (Koscica et al., 2004). LPS is associated with the release of ET-1 (Douthwaite et al., 2003; Forni et al., 2005; Wahl et al., 2005), and blocking formation of this mediator may limit the inflammatory response that triggers premature delivery (Koscica et al., 2004). This finding suggests that phosphoramidon may also protect the lung from LPS-mediated injury.

To test this hypothesis, our laboratory undertook a series of experiments involving treatment of hamsters with either intraperitoneal or aerosolized phosphoramidon prior to induction of acute pulmonary injury by intratracheal administration of lipopolysaccharide. The anti-inflammatory effect of phosphoramidon was assessed by measuring various parameters of lung injury, including morphological changes, leukocyte content of bronchoalveolar lavage fluid (BALF), TNFR1 expression by BALF macrophages, and alveolar septal cell apoptosis. The results indicate that both intraperitoneal and aerosolized administration of phosphoramidon significantly decrease LPS-induced lung injury, and may therefore be potentially useful for the treatment of acute inflammatory lung diseases in humans.

### Methods

### Experimental Design:

To determine the effects of phosphoramidon on LPS-induced lung injury, Syrian hamsters were pretreated with either: 1) intraperitoneal (i.p.) phosphoramidon one hour prior to intratracheal (i.t.) instillation of LPS, or 2) nebulized phosphoramidon immediately prior to i.t. instillation of LPS. Controls were treated with: 1) intraperitoneal (i.p.) PBS followed by i.t. LPS (positive control), 2) nebulized water, followed by i.t. LPS (positive control), 3) no i.p. injection, followed by i.t. PBS (negative control), or 4) nebulized water followed by i.t. PBS (negative control).

Five hours after instillation of LPS, the animals were sacrificed and pulmonary inflammation was determined by measuring: 1) lung histology, 2) neutrophils recovered from BALF, 3) TNFR1-positive BALF macrophages, and 4) alveolar septal cell apoptosis.

Treatment with Phosphoramidon:

Animals were treated with phosphoramidon (Sigma-Aldrich, St Louis, Mo.), either intraperitoneally or intratracheally via nebulization. To examine the effects of intraperitoneal (i.p.) administration, animals were injected with 0.5 mg of phosphoramidon, dissolved in 0.5 ml of PBS. For the nebulization studies, animals were placed in an exposure chamber (28×19×15 in) and exposed for 1 hr to a 0.1 percent solution of phosphoramidon. The aerosolized phosphoramidon was delivered through a ceiling port via a Misty-Ox® nebulizer (Vital Signs, Totowa, N.J.) attached to an air compressor with 25 mg of phosphoramidon dissolved in distilled water. Negative pressure was applied by a blower attached to a secondary outflow port to insure proper circulation of the aerosol. Particle size analysis carried out by Teague Enterprise, Davis Calif. confirmed that the aerodynamic diameter of particles delivered by the Misty-Ox® nebulizer was less than 2 microns, allowing distribution to the distal lung.

Treatment with LPS:

One hr following i.p. or immediately after aerosol administration of phosphoramidon, animals were anesthetized by i.p. injection of 0.4 ml pentobarbital (90 mg/kg) in PBS and instilled intratracheally with 0.5 mg/ml lipopolysaccharide (Sigma-Aldrich, At Louis, Mo.). The solution was delivered via the trachea with a 27-gauge needle attached to a 1-ml syringe. Following instillation, the incision was closed with metal clips.

### Histological Studies:

Animals were euthanized via intraperitoneal (i.p) injection of 0.7 ml of pentobarbital (240 mg/kg), and the lungs were fixed in situ for several hrs with 10% neutral-buffered formalin at a pressure of 20 cm H₂O. Both the lungs and the heart were then removed from the thorax as a single block and fixed in formalin for an additional 48 hrs. After dissecting off the extrapulmonary structures, the lungs were cut into random pieces and entirely submitted for histological processing. Tissue sections were examined with the light microscope after staining with hematoxylin and eosin.

### Determination of Lung Injury:

Tissue sections were graded for injury (inflammatory index), using the following scale based on previously published criteria: (0) no reaction in alveolar walls, (1) diffuse reaction in alveolar walls, with no thickening of the interstitium, (2) diffuse presence of inflammatory cells in alveolar walls with slight thickening of the interstitium, (3) moderate interstitial thickening accompanied by inflammatory cell infiltrates, and (4) interstitial thickening involving more than half of the microscopic field. Results were expressed as an average of 50 microscopic fields.

### Bronchoalveolar Lavage:

Following euthanasia with i.p. pentobarbital, lungs were lavaged 3× with PBS, using 1 aliquot of 3 ml followed by 2 aliquots of 2 ml each. The lavage fluid was centrifuged and the cells were resuspended in PBS. Total leukocyte counts were determined with a hemocytometer. Differential counts were obtained on cytospin preparations treated with Wright's stain.

### Immunocytochemistry of TNFR1:

Cytospin preparations were fixed with ethanol and permeated with an alcohol: acetic acid (2:1) mixture. Endogenous peroxidase activity was quenched with 0.3% H₂O₂ and non-specific binding was blocked with goat serum. The samples were then incubated at room temperature with rabbit anti-mouse TNFR1 polyclonal antibody (Stressgen, Victoria, BC) for 30 minutes, washed and further incubated with biotinylated goat anti-rabbit antibody or rabbit anti-rat antibody for 1 hour at room temperature. After incubation with ABC reagent for 1 hour, the samples were stained with 3, 3′ diaminobenzidine substrate in the dark and counterstained with methyl green. Positive staining for TNFR1 was expressed as a percentage of total macrophages examined.

### TUNEL Assay of Apoptosis:

Tissue sections were digested with 20 μg/ml proteinase K at room temperature, washed, and treated with 0.3% hydrogen peroxide in PBS to quench endogenous peroxidase. After incubation with TdT enzyme at 37° C. for 1 hour, the samples were exposed to anti-digoxigenin conjugate at room temperature for 30 minutes. They were then stained with DAB peroxidase substrate and counterstained with methyl green. Twenty-five high-power (400×) microscopic fields were examined and the results were expressed as mean number of TUNEL-positive cells per 10 high-power fields.

### Statistical Analysis:

The Newman-Keuls multiple comparisons test was used to determine statistically significant differences among the treatment groups (p<0.05).

### Results

### Effect of Phosphoramidon on Lipopolysaccharide-Induced Lung Injury:

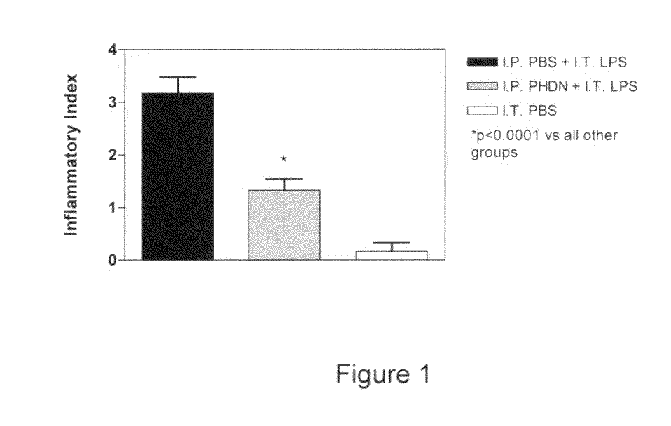

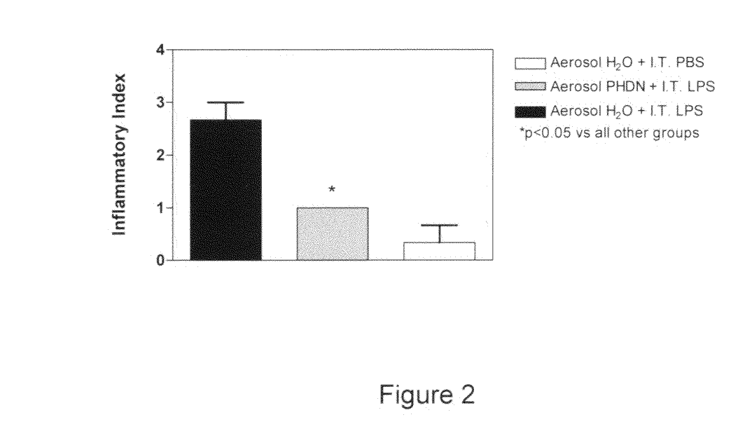

Pretreatment with either i.p. or aerosolized phosphoramidon significantly reduced LPS-induced pulmonary inflammatory changes compared to positive controls. Hamsters treated with i.p. phosphoramidon and i.t. LPS had a mean inflammatory index of 1.3 compared to 3.1 for animals receiving i.p. PBS and LPS (p<0.0005) (FIG. 1). Hamsters treated with aerosolized phosphoramidon and i.t. LPS had mean inflammatory index of 1.0 compared to 2.6 for animals receiving aerosolized water and i.t. LPS (p<0.05) (FIG. 2).

### BAL Measurements:

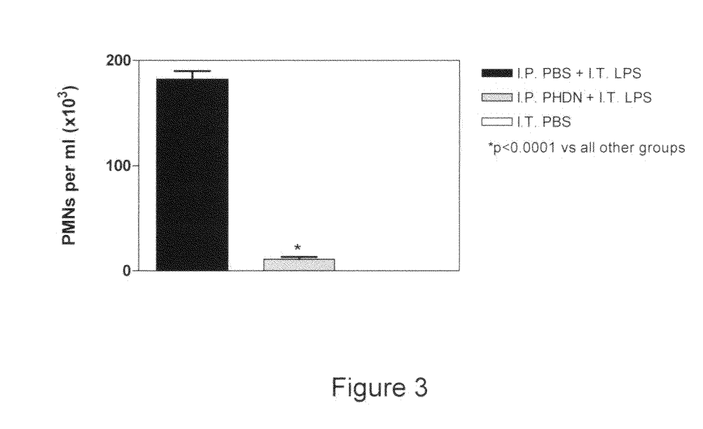

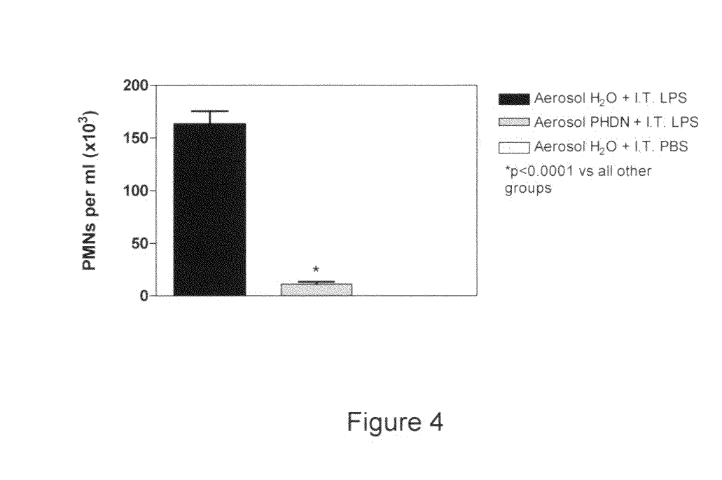

Pretreatment with either i.p. or aerosolized phosphoramidon significantly lowered the total number of BALF neutrophils compared to positive controls. Hamsters treated with i.p. phosphoramidon and i.t. LPS had a mean total of 1.1×10⁴ cells/ml compared to 18.2×10⁴ cells/ml for animals receiving i.p. PBS and LPS (p<0.0001) (FIG. 3). Hamsters treated with aerosolized phosphoramidon and i.t. LPS averaged 1.1×10⁴ cells/ml compared to 16.4×10⁴ cells/ml for animals receiving aerosolized water and i.t. LPS (p<0.0001) (FIG. 4).

### TNFR1 Measurements:

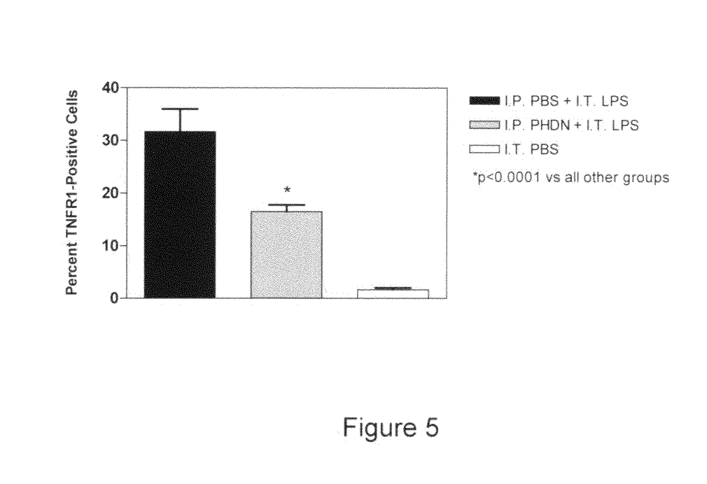

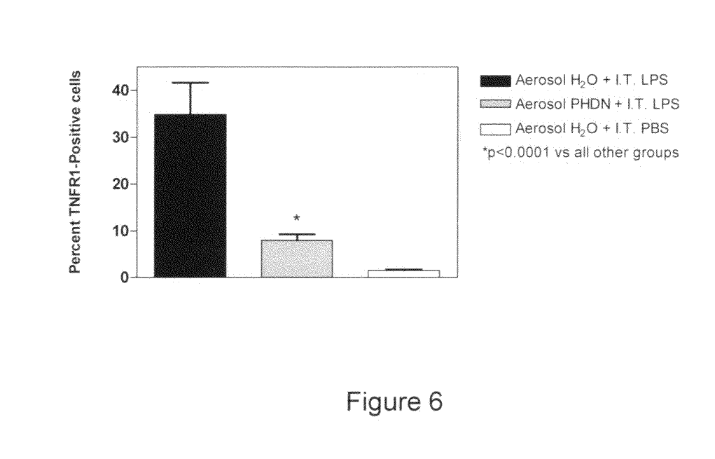

Pretreatment with either i.p. or aerosolized phosphoramidon significantly reduced the expression of TNFR1 by macrophages compared to positive controls. Hamsters treated with i.p. phosphoramidon and i.t. LPS had 16.5% TNFR1-positive cells compared to 31.6% for animals receiving i.p. PBS and i.t. LPS (p<0.01) (FIG. 5). Hamsters treated with aerosolized phosphoramidon and i.t. LPS had 8.0% TNFR1-positive cells compared to 34.8% for animals receiving aerosolized water and i.t. LPS (p<0.05) (FIG. 6).

### Lung Cell Apoptosis:

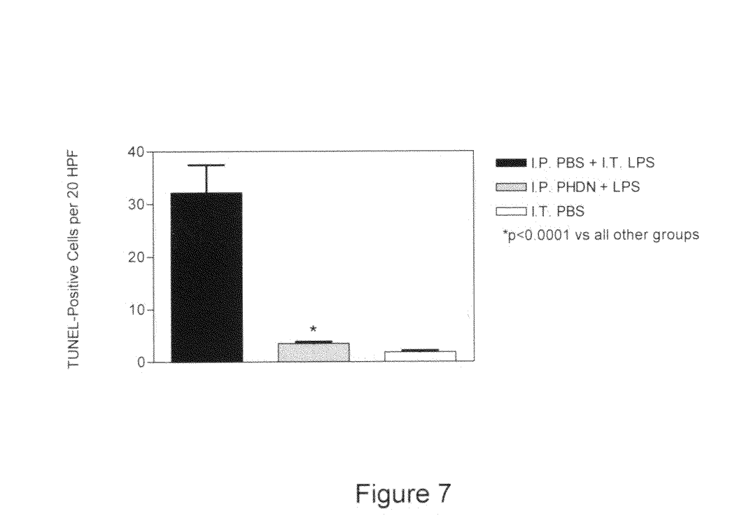

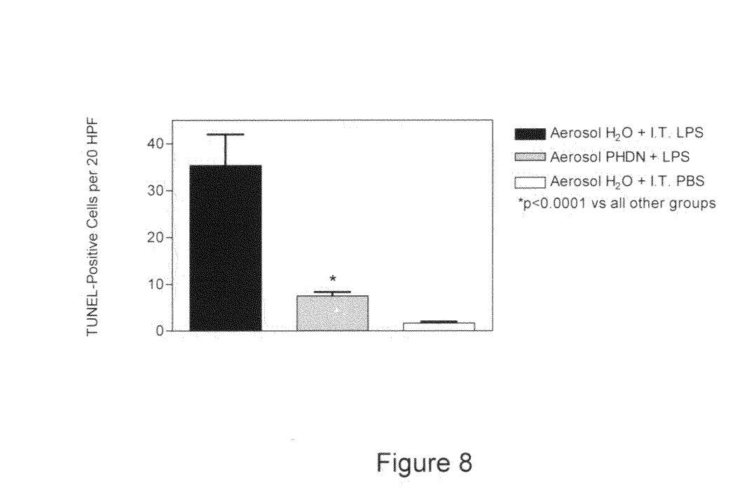

Pretreatment with either i.p. or aerosolized phosphoramidon prior significantly reduced the number of TUNEL-positive alveolar septal cells compared to positive controls. Hamsters treated with i.p. phosphoramidon and i.t. LPS had had 7.5 labeled cells per 10 high-power microscopic fields compared to 35.3 for animals receiving i.p. PBS and i.t. LPS (p<0.05) (FIG. 7). Hamsters treated with aerosolized phosphoramidon and i.t. LPS had 3.5 labeled cells per 10 high-power microscopic fields compared to 32.1 for animals receiving aerosolized water and i.t. LPS (p<0.0005) (FIG. 8).

### Discussion

Phosphoramidon has been characterized as a potent and specific inhibitor of a zinc metalloproteinase (ECE) responsible for converting a 39-amino acid peptide, “big” ET-1, to the 21-amino acid peptide, ET-1 (Donahue et al., 2006; Wahl et al., 2005; Ikegawa et al., 1991; Fawzi et al., 1994). Its ability to inhibit ECE has rendered it a valuable tool in the study of hypertension, stroke, and diseases of the kidney (Hassan et al., 1997; Keller et al., 1996). Phosphoramidon has also been shown to reduce tissue injury related to compromised blood flow. (Park et al., 2001; Aguilar et al., 2000).

Recently, the anti-inflammatory effects of phosphoramidon have also been examined, most notably in a mouse model of preterm delivery involving the use of LPS (Koscica et al., 2004). That study provided the rationale for the current investigation of the effect of phosphoramidon on LPS-induced acute lung injury. Using either an intraperitoneal or intratracheal route of administration, it was shown that phosphoramidon can significantly attenuate the effects of LPS on the lung.

Despite the fact that the animals received a much lower total dose of phosphoramidon via aerosolization, the results of direct exposure of the lung to this agent over a period of 1 hr were shown to be comparable to intraperitoneal administration. This finding is particularly relevant to the potential use of phosphoramidon as a treatment for human lung injury.

While the mechanism responsible for the anti-inflammatory effect of phosphoramidon remains unclear, it presumably involves a reduction in ET-1, a mediator that plays a central role in LPS-induced tissue injury (Wahl et al., 2005; Zaedi et al., 2006; Waneeek et al., 2000). Studies have shown that LPS stimulates the synthesis of ET-1 by cultured monocytes and endothelial cells (Wahl et al., 2005; Ebihara et al., 1998; Liu et al., 1997; Forni et al., 2005), and is also a potent inducer of ET-1 synthesis in vivo (Forni et al., 2005; Ohta et al., 1990).

The inflammatory modulator functions of ET-1 are primarily mediated by ET_{A} receptor activity (Fagan et al., 2001; Donaubauer et al., 2006; Hay et al., 2001). The binding of ET-1 to these receptors is responsible for causing release of inflammatory mediators such as leukotrienes and platelet activating factor (Fagan et al., 2001; Donaubauer et al., 2006). ET-1 may also be involved in the recruitment of inflammatory cells by enhancing the expression of intercellular adhesion molecule-1, vascular cell adhesion molecule-1, and E-selectin (Sampaio et al., 2000, 2004; Helset et al., 1996; Farre et al., 1993; Filep et al., 1993).

The marked reduction in BALF neutrophils following treatment with phosphoramidon suggests that the anti-inflammatory activity of this agent may be largely dependent on blocking the effects of ET-1 on the vascular compartment. However, other factors may also be responsible for this finding, including decreased expression of macrophage TNFR1. The loss of this receptor may limit the activity of TNF-alpha, a cytokine that can activate a number of macrophage proinflammatory genes (Thomas, 2001), including those responsible for the synthesis of metalloproteinases that play a role in the migration of inflammatory cells to the lung.

The reduced rate of alveolar septal cell apoptosis seen in the phosphoramidon-treated lungs may also be related to a decrease in ET-1. Binding of ET-1 to ET_{A} results in the coupling of G proteins that induce apoptosis. (Fagan et al., 2001; Forni et al., 2005). While the effect of apoptosis on the lungs of LPS-treated animals remains unclear, the loss of alveolar septal cells by this process has previously been shown to play a role in the pathogenesis of pulmonary emphysema (Kasahara et al., 2000). This finding may be relevant to the current studies, since repeated intratracheal administration of LPS can result in airspace enlargement (Stolk et al., 1992).

Regarding the role of phosphoramidon in attenuating inflammation, a conflicting issue is its potent inhibitory effect on NEP. Inhibition of this enzyme has been shown to accentuate pulmonary inflammation (Day et al., 2005). However, our observations indicate that the primary effect of phosphoramidon involves the blockade of ECE and a reduction in the acute inflammatory response. Consequently, ECE inhibitors (and endothelin receptor anatgonists) may be potentially useful agents for the treatment of inflammatory lung disease in humans.

### REFERENCES

1. Aguilar, A. M. A., Revert, F., Moya, A., Beltran, J., Garcia, J., Martin, E. S., Sancho, S., and Such, L. (2000). Intravenous BQ-123 and phosphoramidon reduce ventricular ectopic beats and myocardial infarct size in dogs submitted to coronary occlusion and reperfusion. General Pharmacology 35, 143-147.
2. Day, A. L., Wick, E., Jordan, T. H., Jaffray, C. E., Bunnett, N. W., Grady, E. F., Kirkwood, K. S. (2005). Neutral endopeptidase determines the severity of pancreatitis-associated lung injury. Journal of Surgical Research 128(1), 21-7.
3. Donahue, M. and Johnston, J. (2006). Preparation of a protected phosphoramidon precursor via an H-Phosphonate coupling strategy. Bioorganic & Medicinal Chemistry Letters 16, 5602-5604.
4. Donaubauer, B., Busch, T., Lachmann, R., Deja, M., Petersen, B., Francis, R., Trager, A., Ebsen, M., Boemke, W., and Kaisers, U. (2006). Low-Dose Inhalation of an Endothelin-A Receptor Antagonist in Experimental Acute Lung Injury: ET-1 Plasma Concentration and Pulmonary Inflammation. Experimental Biology and Medicine 231, 960-966.
5. Douthwaite, J. A., Lees, D. M., Corder, R. (2003). A role of increased mRNA stability in the induction of endothelin synthesis by lipopolysaccharide. Biochemical Pharmacology 66, 589-594.
6. Ebihara, I., Nakamura, T., Shimada, N., Shoji, H., and Koide, H. (1998). Effect of hemoperfusion with polymyxin B-immobilized fiber on plasma endothelin-1 and endothelin-1 mRNA in monocytes from patients with sepsis. American Journal of Kidney Diseases 32, 953-961.
7. Fagan, K., McMurtry, I., and Rodman, D. (2001). Role of endothelin-1 in lung disease. Respir Res 2, 90-101.
8. Farre, A. L., Riesco, A., Espinosa, G., Digiuni, E., Cernadas, M. R., Alvarez, V., Monton, M., Rivas, F., Gallego, M. J., Egido, J., Casado, S., and Caramelo, C. (1993). Effect of endothelin-1 on neutrophil adhesion to endothelial cells and perfused heart. Circulation 88, 1166-1171.
9. Fawzi, A. B., Cleven, R. M., and Wright, D. L. (1994). A Rapid and Selective Endothelin-Converting Enzyme Assay: Characterization of a Phosphoramidon-Sensitive Enzyme from Guinea Pig Lung Membrane. Analytical Biochemistry 222, 342-350.
10. Filep, J. G., Sirios, M. G., Foldes-Filep, E., Rousseau, A., Plante, G. E., Fournier, A., Yano, M., and Sirios, P. (1993). Enhancement by endothelin-1 of microvascular permeability via the activation of ETA receptors. British Journal of Pharmacology 109, 880-886.
11. Forni, M., Mazzola, S., Ribeiro, L. A., Pirrone, F., Zannoni, A., Bernardini, C., Bacci M. L., and Albertini, M. (2005). Expression of endothelin-1 system in a pig model of endotoxic shock. Regulatory Peptides 131, 89-96.
12 Hassan, M., Kashimura, H., Matsumaru, K., Nakahara, A., Fukutomi, H., Muto, H., Goto, K., and Tanaka, N. (1997). Phosphoramidon, An Endothelin Converting Enzyme Inhibitor Attenuates Local Gastric Ischemia-Reperfusion Injury in Rats. Life Sciences 61, 141-147.
13. Hay, D. W. P., and Compton, C. H. (2001). Endothelin Antagonists. Prog Respir Res. Basel, Karger 31, 141-144.
14. Helset, E., Lindal S., Olsen, R., Myklebust, R., and Jorgensen, L. (1996). Endothelin-1 causes sequential trapping of platelets and neutrophils in pulmonary microcirculation in rats. American Journal of Physiology 271, L538-L546.
15. Hisaki, K., Matsumura, Y., Maekawa, H., Fujita K., Takaoka, M., and Morimoto, S. (1994). Conversion of Big ET-1 in the rat lung: role of phosphoramidon-sensitive endothelin-1-converting enzyme. Am. J. Physio. 266, H422-H428.
16. Ikegawa, R., Matsumura, Y., Tsukahara, Y., Takaoka, M. And Morimoto, S. (1991). Phosphoramidon inhibits the generation of endothelin-1 from exogenously applied big endothelin-1 in cultured vascular endothelial cells and smooth muscle cells. Federation of European Biochemical Societies 293, 45-48.
17. Kasahara, Y., Tuder, R. M., Taraseviciene-Stewart, L., Le Cras, T. D., Abman, S., Hirth, P. K., Waltenberger, J., Voelkel, N. F. (2000). Inhibition of VEGF receptors causes lung cell apoptosis and emphysema. Journal of Clinical Investigation 106, 1311-1319.
18. Keller, P. M., Lee C. P., Fenwick, A. E., Atkinson, S. T., Elliot, J. D., and Wolf, W. E. De., Jr. (1996). Endothelin-Converting Enzyme: Substrate Specificity and Inhibition by Novel Analogs of Phosphoramidon. Biochemical and Biophysical Research Communications 223, 372-378.
19. Koscica, K. L., Sylvestre, G., Reznik, S. E. (2004). The effect of phosphoramidon on inflammation-mediated preterm delivery in a mouse model. American Journal of Obstetrics and Gynaecology 190, 528-531.
20. Laporte, J., and Sirois, P. (1997). Phosphoramidon and thiorphan suppress the generation of endothelin (ET) from exogenous big-endothelin by guinea pig Clara cells. Regulatory Peptides 68, 105-109.
21. Liu, B., Zhou, J., Chen, H., Wang, D., Hu, D., Wen, Y., and Xiao, N. (1997). Expression and cellular location of endothelin-1 mRNA in rat liver following endotoxemia. Chinese Medical Journal 110, 932-935.
22. Matsumaru, K., Kashimura H., Hassan, M., Nakahara, A., Goto, K., Fukutomi, H., Muto, H., and Tanaka, N. (1998). Phosphoramidon, An Inhibitor of Endothelin-Converting Enzyme, Prevents Indomethacin-Induced Gastric Mucosal Damage in Rats. Life Sciences 62, 79-84.
23. Ohta, K., Hirata, Y., Imai, T., Kanno, K., Emori, T., Shichiri, M., et al. (1990). Cytokine-induced release of endothelin-1 from porcine renal epithelial cell line. Biochemical and Biophysical Research Communications 169, 578-584.
24. Park, L., and Thornhill, J. (2001). Phosphoramidon-sensitive endothelin-converting enzymes modulate cerebral blood flow and neural damage of hypoxic rats. Neuroscience letters 301, 95-98.
25. Sampaio, A. L. F., Rae, G. A., and Henriques, M. G. (2004). Journal of Leukocyte Biology 76, 210-216.
26. Sampaio, A., Rae, G., and Henriques, M. (2000). Participation of endogenous endothelins in delayed eosinophil and neutrophil recruitment in mouse pleurisy. Inflammation Research 49, 170-176.
27. Stolk, J., Rudolphus, A., Davies, P., Osing a, D., Dijkman, J. H., Agarwal, L., Keenan, K. P., Fletcher, D., Kramps, J. A. (1992). Induction of emphysema and bronchial mucus cell hyperplasia by intratracheall instillation of lipopolysaccharide in the hamster. Journal of Pathology 167, 349-356.
28. Thomas, P. S. (2001). Tumor necrosis Factor-α, The role of this multifunctional cytokine in asthma. Immunology and Cell Biology 79, 132-140.
29 Wahl, J. R., Goetsch, N. J., Young, H. J., Maanen, R. J. V., Johnson, J. D., Pea, A. S., and Brittingham, A. (2005). Murine Macrophages Produce Endothelin-1 After Microbial Stimulation. Experimental Biology and Medicine 230, 652-658.
30 Waneeek, M., Weitzberg, E., Rudchill, A., Oldner, A. (2000). The endothelin system in septic and endotoxic shock. European Journal of Pharmacology 407, 1-15.
31 Zaedi, S., Jesmin, S., Maeda, S., Shimojo, N., Yamaguchi, I., Goto, K., and Miyauchi, T. (2006). Alterations in Gene Expressions Encoding PreproET-1 and NOS in Pulmonary Tissue in Endotoxemic Rats. Experimental Biology and Medicine 231, 992-996.

What is claimed is:

1. A method of treating a respiratory disorder in a mammal that comprises intratracheal administration of an endothelin-converting enzyme (ECE) inhibitor.

2. A method of claim 1, wherein the ECE inhibitor is phosphoramidon.

3. A method of claim 1, wherein the respiratory disorder is one of the following: emphysema, asthma, bronchitis, bronchiectasis, pneumonia, cystic fibrosis, interstitial pulmonary fibrosis, adult respiratory distress syndrome, neonatal respiratory distress syndrome, bronchopulmonary dysplasia, persistent pulmonary hypertension of the newborn, or neoplasia.

4. A method of claim 1, wherein the mammal is an adult human or neonate.

5. A method of claim 1, wherein the intratracheal administration is performed by nebulization.

6. A method of claim 1, wherein the ECE inhibitor is isolated from a natural source.

7. A method of claim 1, wherein the ECE inhibitor is produced by a bioprocess, such as fermentation.

8. A method of claim 1, wherein the ECE inhibitor is chemically synthesized.

9. A method of claim 1, wherein the ECE inhibitor is administered with a carrier, such as DMSO, an alcohol, or water.

10. A method of claim 1, wherein the effective amount of the ECE inhibitor is from about 10 μg/kg/day to about 1 mg/kg/day.

11. A method of treating a respiratory disorder in a mammal that comprises intratracheal administration of an endothelin receptor antagonist.

12. A method of claim 11, wherein the endothelin receptor antagonist is one of the following: bosentan, tezosentan, sitaxsentan, atrasentan, darusentan, clazosentan, or BQ-123.

13. A method of claim 11, wherein the respiratory disorder is one of the following: emphysema, asthma, bronchitis, bronchiectasis, pneumonia, cystic fibrosis, interstitial pulmonary fibrosis, adult respiratory distress syndrome, neonatal respiratory distress syndrome, bronchopulmonary dysplasia, persistent pulmonary hypertension of the newborn, or neoplasia.

14. A method of claim 11, wherein the mammal is an adult human or neonate.

15. A method of claim 11, wherein the wherein the intratracheal administration is performed by nebulization.

16. A method of claim 11, wherein the endothelin receptor antagonist is isolated from a natural source.

17. A method of claim 11, wherein the endothelin receptor antagonist is produced by a bioprocess, such as fermentation.

18. A method of claim 11, wherein the endothelin receptor antagonist is chemically synthesized.

19. A method of claim 11, wherein the endothelin receptor antagonist is administered with a carrier, such as DMSO, an alcohol, or water.

20. A method of claim 11, wherein the effective amount of the endothelin receptor antagonist is from about 10 μg/kg/day to about 1 mg/kg/day.
